(19) United States
(12) Patent Application Publication
(10) Pub. No.: US 2025/0312374 A1
(43) Pub. Date: Oct. 9, 2025

(54) ELECTROLYTES FOR ADDRESSING LOW IRON

(71) Applicant: Jake Rabinowitz, Queens, NY (US)

(72) Inventor: Jake Rabinowitz, Queens, NY (US)

(21) Appl. No.: 18/626,432

(22) Filed: Apr. 4, 2024

(51) Int. Cl. A61K 33/26; A61K 9/00; A61K 9/107; A61K 31/07; A61K 31/095; A61K 31/12; A61K 31/197; A61K 31/205; A61K 31/355; A61K 31/375; A61K 31/525; A61K 31/593; A61K 31/714; A61K 33/14

(52) U.S. Cl. CPC A61K 33/26; A61K 9/0053; A61K 9/107; A61K 31/07; A61K 31/095; A61K 31/12; A61K 31/197; A61K 31/205; A61K 31/355; A61K 31/375; A61K 31/525; A61K 31/593; A61K 31/714; A61K 33/14

(57) ABSTRACT

In some embodiments, a hydration powder comprises an electrolyte blend, the electrolyte blend comprising an iron component present at a mass ratio of at least 0.15%, a sodium component present at a mass ratio of at least 9.0%, and a potassium component present at a mass ratio of at least 9.0%. In some embodiments, the iron component comprises ferric glycinate. In some embodiments, a packaged electrolyte blend comprises an iron component present at a mass of at least 2 mg or at least 10 mg, a sodium component present at a mass of at least 400 mg, and a potassium component present at a mass of at least 400 mg. In some embodiments, the electrolyte blends further comprise any one or more of vitamins B12, B9, B2, C, A, or E.

### BACKGROUND

The present disclosure generally relates to nutrition solutions (e.g., for administering electrolyte or nutrient blends with iron). In some embodiments, the nutrition solutions increase the absorption and tolerance of orally ingested iron.

### SUMMARY

Iron is an essential nutrient and low iron is the world's most common nutrient deficiency. A typical solution to low iron is to administer very high doses of a poorly absorbed iron supplement. This solution is inadequate for many people, including those who cannot tolerate the high doses of iron, have absorption issues, and/or have compounding nutrient deficiencies contributing to their low iron state. Accordingly, nutrient emulsions are disclosed herein for improved iron absorption and tolerance.

In a first embodiment, a hydration powder comprises an electrolyte blend, the electrolyte blend comprising an iron component present at a mass ratio of at least 0.15%, a sodium component present at a mass ratio of at least 9.0%, and a potassium component present at a mass ratio of at least 9.0%.

In a first implementation of the first embodiment, the iron component comprises ferric glycinate, the ferric glycinate at a mass ratio of at least 1.05%, the sodium component comprises sodium chloride, the sodium chloride at a mass ratio of at least 24.1%, and the potassium component comprises potassium citrate, the potassium citrate at a mass ratio of at least 74.7%.

In a first aspect of that first implementation, the electrolyte blend further comprises a magnesium component present at a mass ratio of at least 0.29%, a calcium component present at a mass ratio of at least 1.8%, a zinc component present at a mass ratio of at least 0.029%, a methylcobalamin component present at a mass ratio of at least 0.00014%, a cholecalciferol component present at a mass ratio of at least 0.00028%, and an ascorbic acid component present at a mass ratio of at least 2.3%.

In a first iteration of that first aspect, the magnesium component comprises magnesium glycinate, the magnesium glycinate at a mass ratio of at least 2.0%, the calcium component comprises calcium citrate, the calcium citrate at a mass ratio of at least 23.5%, and the zinc component comprises zinc gluconate, the zinc gluconate at a mass ratio of at least 0.15%, such that the mass ratio of the ferric glycinate is reduced to at least 0.7%, the mass ratio of the sodium chloride is reduced to at least 17.0%, and the mass ratio of the potassium citrate is reduced to at least 53.0%.

In a second implementation of that first embodiment, the electrolyte blend further comprises a methylcobalamin component present at a mass ratio of at least 0.00019%, a cholecalciferol component present at a mass ratio of at least 0.00039%, an ascorbic acid component present at a mass ratio of at least 3%, a beta carotene component present at a mass ratio of 0.3%, a tocopheryl acetate component present at a mass ratio of at least 0.4%, a riboflavin component present at a mass ratio of at least 0.02%, and a folate component present at a mass ratio of at least 0.007%.

In a first aspect of that second implementation, the electrolyte blend further comprises a taurine component present at a mass ratio of at least 0.18%, and a carnitine component present at a mass ratio of at least 0.18%.

In a third implementation of that first embodiment, the electrolyte blend further comprises at least one acid, the at least one acid comprising at least one of citric acid, malic acid, or tartaric acid, at least one sweetener, the at least one sweetener comprising at least one of sugar, allulose, or monk fruit extract, at least one natural flavor, and methoxatin.

In a second embodiment of the present disclosure, a packaged electrolyte blend comprises an iron component present at a mass of at least 2 mg, a sodium component present at a mass of at least 400 mg, and a potassium component present at a mass of at least 400 mg.

In a first implementation of the second embodiment, the packaged electrolyte blend is individually arranged within a sealed container.

In a first aspect of that first implementation, the sealed container further comprises at least 6 ounces of water.

In a second aspect of that first implementation, the sealed container further comprises at least 1 ounce of gel.

In a first iteration of that second aspect, the at least 1 ounce of gel comprises the iron component, the sodium component, the potassium component, and a carbohydrate component.

In a second implementation of the second embodiment, the packaged nutrient blend is individually arranged as a dissolvable tablet.

In a third implementation of the second embodiment, the iron component is present at a mass of at least 10 mg, wherein the iron component comprises at least one of ferric glycinate or ferrous glycinate.

In a fourth implementation of the second embodiment, the iron component is present at a mass of at least 10 mg, wherein the iron component comprises at least one of iron protein succinylate or heme iron polypeptide.

In a fifth implementation of the second embodiment, the electrolyte blend further comprises a methylcobalamin component present at a mass ratio of at least 0.01 mg, a cholecalciferol component present at a mass ratio of at least 0.02 mg, and an ascorbic acid component present at a mass ratio of at least 160 mg.

In a first aspect of that fifth implementation, the electrolyte blend further comprises a beta carotene component present at a mass of at least 0.9 mg, a tocopheryl acetate component present at a mass of at least 20 mg, a riboflavin component present at a mass of at least 1.3 mg, and a folate component present at a mass of at least 0.4 mg. In a sixth implementation of the second embodiment, the electrolyte blend further comprises a taurine component present at a mass of at least 100 mg, a carnitine component present at a mass of at least 100 mg, and a methoxatin component present at a mass of at least 5 mg. In a seventh implementation of the second embodiment, the electrolyte blend further comprises at least one amino acid present at a mass of at least 100 mg, the at least one amino acid comprising at least one of glutamine, beta-alanine, methionine, histidine, leucine, or threonine. In a eighth implementation of the second embodiment, the electrolyte blend further comprises at least one antioxidant present at a mass of at least 100 mg, the at least one antioxidant comprising at least one of glutathione, methoxatin, docosahexaenoic acid, lycopene, carotene, or resveratrol.

### BRIEF DESCRIPTION OF THE DRAWINGS

Within the following description is a discussion of the following figures, which are provided as some illustrative embodiments of the present disclosure. Accordingly, the figures should be viewed as example embodiments and not limitations of the present disclosure.

FIG. 1 shows a first nutrient emulsion for improved iron absorption, according to some embodiments of the present disclosure;

FIG. 2 shows a second nutrient emulsion for improved iron absorption, according to some embodiments of the present disclosure;

FIG. 3 shows a third nutrient emulsion for improved iron absorption, according to some embodiments of the present disclosure;

FIG. 4 shows phases of a nutrient emulsion for improved iron absorption, according to some embodiments of the present disclosure;

FIG. 5 shows interfacial components of a nutrient emulsion for improved iron absorption, according to some embodiments of the present disclosure;

FIG. 6 shows factors contributing to iron deficiency, according to some embodiments of the present disclosure; and

FIG. 7 shows an illustrative flowchart of a method for manufacturing a nutrient emulsion for providing bioavailable iron.

### DETAILED DESCRIPTION

Iron supports normal biological activity including regulation of circulatory, respiratory, neurological, hormonal, digestive, musculoskeletal, immunological, and other functions. When orally ingested, the bioavailability of the iron varies according to at least the molecular state of the iron, an individual's physiology, and the possible presence of other orally ingested nutrients that enhance or inhibit iron absorption. As used herein, the “bioavailability” or “absorption” of iron may refer to the fraction of ingested elemental iron that is used to synthesize blood or stored in ferritin. Iron absorption may be inhibited due to anti-nutrients (e.g., nutrients that bind to the iron and cause it pass through the gastrointestinal (GI) tract without being absorbed) making iron unavailable at a primary absorption site (e.g., the duodenum or the jejunum), at a secondary absorption site (e.g., the colon), or at other absorption sites. Iron absorption may be enhanced (e.g., compared to orally ingesting iron without any other nutrients) by pro-nutrients that make iron available for absorption (e.g., by chelating ferric iron) or for storage (e.g., by providing amino acids for synthesizing ferritin).

In humans, low iron may describe any state where normal biological functions are disrupted due to a deficiency of in vivo iron availability and/or utilization, which may reduce the prevalence of healthy blood, disrupt the flow of oxygen to many organs (thereby inhibiting their normal function), inhibit biophysical processes for which iron acts as a direct or indirect cofactor, or cause other adverse effects. Low iron can cause symptoms including, but not limited to, fatigue, depression, weakness, shortness of breath, hormone dysregulation, dizziness, photopsia, amplified ecchymosis, restless legs, pale skin, brittle nails, hair loss, poor appetite, feeling cold, odd cravings, mouth sores, and gastrointestinal dysregulation.

Despite the myriad symptoms of low iron, this condition remains extremely prevalent in the US and worldwide. Anemia, which is often a manifestation of low iron (i.e., iron deficiency anemia) is estimated to affect ˜6% of the US population and ˜33% of the global population. These values understate the prevalence of low iron, because low iron does not necessarily result in anemia. Notably, low iron symptoms initiate in many patients whose iron levels are above clinically recognized thresholds for diagnosing low iron.

Low iron is prevalent in many groups, including people who are pregnant or nursing (due to providing iron to the growing baby), people who experience regular blood loss including due to menstruation (due to requiring more iron for erythropoiesis), children (due to poor diet and/or lacking iron stores), teens (due to rapid growth), elders (due to poor diet and/or inflammation), people who are ill (due to infection, inflammation, internal bleeding, gastrointestinal dysfunction, reactions to medication, or any combination thereof), runners (due to inflammation and hemolysis), vegetarians/vegans (due to lack of dietary intake), and bariatric patients (due to removal of small intestine).

For individuals with low iron, iron supplementation may be helpful to raise iron levels. However, iron supplementation often causes adverse side effects including constipation, nausea, bloating, indigestion, and/or diarrhea. These adverse effects of iron supplementation may outweigh the benefits of improved iron levels, such that many people live with chronically low iron rather than maintain supplementation.

Anemia, particularly iron deficiency anemia, is a condition that is diagnosed by a lack of healthy red blood cells. When an individual's low iron status has progressed to anemia, additional nutrients may be required to restore normal iron and physiological status. Among these other nutrients are vitamins B9 (e.g., folate or a form thereof) B12 (e.g., cobalamin or a form thereof), C (e.g., ascorbate or a form thereof), D (e.g., calciferol or a form thereof), and E (e.g., tocopherol or a form thereof), selenium, and magnesium.

There remains a massive and urgent need for nutrition packages that administer iron in a form that maximizes bioavailability, ensures tolerance, and simultaneously addressing other dietary (e.g., additional nutrient deficiencies) and physiological (e.g., causes of iron malabsorption) conditions affecting the low iron individual.

In accordance with embodiments of the present disclosure, nutrient emulsions, nutrient blends, and electrolyte blends are provided for delivering bioavailable iron. Bioavailable iron may be delivered in a chelated form that readily dissolves in water. During administration, the water may be ingested. During preparation, the water may be incorporated into the water phase of a water-in-oil emulsion (e.g., for subsequent administration). In some embodiments, the emulsions have more than two phases (e.g., oil-in-water-in-oil emulsions, water-in-oil-in-water-in-oil emulsions, and emulsions with greater number of phases). Within these nutrient emulsions having two or more phases, any outer phase (such as the oil of a water-in-oil emulsion) may include one or more discrete units of its respective inner phase (e.g., the water of a water-in-oil emulsion).

Due to being dissolved in a water phase and encapsulated by an oil phase of the emulsion, the ingested iron may be at least partially prevented from releasing in stomach fluid and/or upper gastrointestinal lining (e.g., areas of the GI tract preceding the small intestine). Thus, the bioavailable iron is less exposed to malabsorption (e.g., by diffusing into cells that do not reside at primary physiological iron absorption sites and thus may be less conditioned to release the iron as needed and/or transport the iron into the labile iron pool). Such malabsorption may also lead to the generation of free radicals via the Fenton reaction, which are toxic to the body. In contrast, the bioavailable iron is delivered, during normal digestive transit, within the emulsion or chelation to the small intestine, where the oil phase or the chelated molecule may be broken down by at least enzymes and microbes. In response to this breakdown, the water-dissolved bioavailable iron is released in the small intestine near iron absorption sites at the proximal jejunum and duodenum.

To further enhance iron absorption, vitamin C may additionally be included in the nutrient blend, electrolyte blend, or water phase of the emulsion with at least an 8:1 molar ratio of vitamin C (i.e., ascorbic acid) to elemental iron. This vitamin C forms a soluble chelate complex with iron, particularly ferric iron, if the iron does get released from its as-sourced carrier molecule (e.g., glycine, gluconate) during emulsion preparation and/or within the digestive tract. This vitamin C-iron chelate is soluble in the alkaline environment of the small intestine, where duodenal iron absorption membranes reduce ferric iron to ferrous iron and then shuttle the ferrous iron inside corresponding cells, thus completing intestinal uptake.

At least one lecithin and at least one fiber (e.g., at least one of gum, pectin, inulin, or oligosaccharides) are additionally included in the nutrient blend or emulsion to stabilize its external interface and its at least one internal phase interface. The ensuing highly stabilized blend or emulsion is protected against de-emulsification during processing, storage, digestion, or any combination thereof. The fiber additionally serves to improve iron bioavailability by providing one or more prebiotic nutrients that support probiotic microbes in the GI tract, as further described below.

In some embodiments, at least one transition metal is included to activate ion channels that uptake iron, including at the proximal jejunum and duodenum.

In some embodiments, at least one antioxidant is included to provide anti-inflammatory effects. Inflammation can initiate immune responses, including expression of hepcidin, that lower iron absorption. Therefore, in some embodiments, antioxidants are included to improve iron absorption.

In some embodiments, at least one nutrient to support the gut microbiome (e.g., a prebiotic fiber, probiotic organism, or a medium-chain fat) is included to regulate immune responses that reduce iron absorption. During the immune response to an infection or related pathogenesis, iron levels may be reduced to “starve out” the pathogenic agent. Therefore, nutrients that support the gut microbiome may improve iron absorption.

In some embodiments, at least one nutrient to support hormone regulation (e.g., inositol, an omega-3 PUFA, or an essential vitamin/mineral) is included to regulate the levels of hormones that regulate iron absorption. For example, hepcidin is a hormone that inhibits iron absorption. Therefore, nutrients that regulate hormone production (e.g., by lowering hepcidin levels) may improve iron absorption.

In some embodiments of the present disclosure, blends or emulsions deliver the iron in a bioavailable package, deliver the iron along with synergistic nutrients that enhance bioavailability, protect the iron from environmental stress prior to administration, stabilize the iron from the time of emulsion formation through digestive transit to iron absorption sites, and co-deliver the iron with other beneficial nutrients.

In the present disclosure, an emulsion is a multi-phase system (i.e., two or more phases) wherein at least one stable particle of a first phase (e.g., water) is encapsulated by a stable particle of a second phase (e.g., oil). In some embodiments, the at least one stable particle of a first phase may further encapsulate one or more stable particles of a third phase (e.g., oil), where the third phase particles may be smaller masses of the second phase material. These encapsulated particles of the third phase may further encapsulate stable particles of a fourth phase, where the fourth phase particles may be smaller masses of the first phase material, and so on. Each stable particle of an emulsion may be any size, and is typically in the range of 10 nm through 100 μm.

In some embodiments of the present disclosure, an emulsion or nutrient blend is formed through a multi-step process including batching nutrients in water and homogenizing the nutrients. The homogenization step incorporates disparate nutrients of the emulsion or nutrient blend into a stable emulsion particle. Nutrients including lecithin and fiber (e.g., gum or pectin) are soluble in both phases of the emulsion and thus stabilize the emulsion particle by bridging internal (e.g., two-phase) and external interfaces of the emulsion. Small carbohydrates (e.g., sugar or allulose) may also reside at or near the interfaces for further stabilization (e.g., as wall materials). Excluding lecithin and fiber, most other nutrients are soluble in one, but not both, of the phases composing the emulsion particle. Therefore, the emulsion is required to deliver diverse water- and oil-soluble nutrients within a single consumable package.

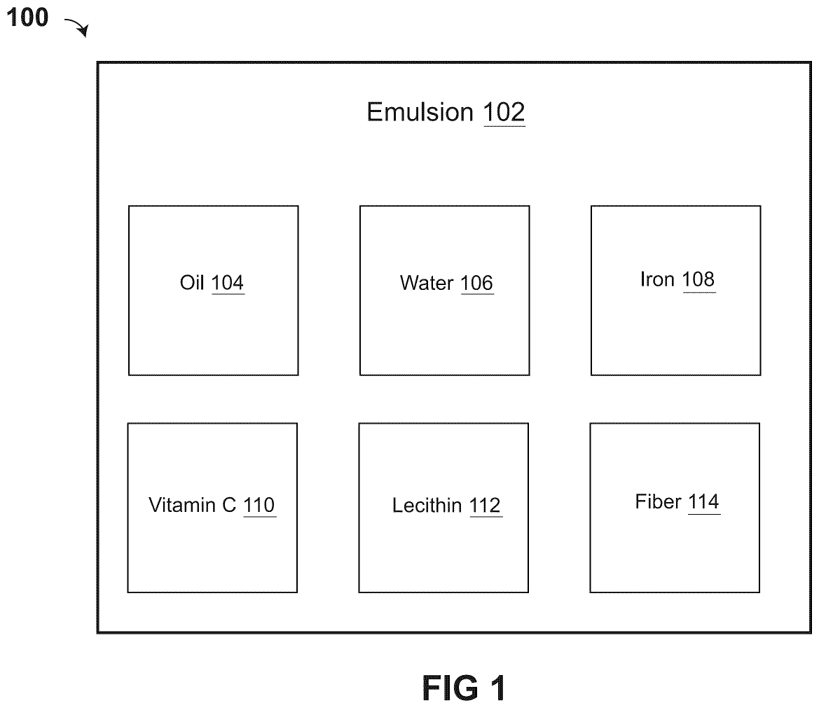

The present subject matter may be better understood with reference to FIGS. 1-7. As used herein, a mass ratio refers to a ratio (e.g., which may be presented as a decimal, where the decimal represents any fraction, or may be presented as a percentage, where the percentage represents the decimal as a fraction of 100) defined as the mass of a respective nutrient (e.g., an electrolyte, vitamin, antioxidant, protein, carbohydrate, fiber, fat, water, a compound comprising a nutrient, or any other suitable nutrient or compound) over the total mass of a nutrient blend (e.g., where the nutrients are combined in an emulsion) an electrolyte blend, or any other suitable composite blend (e.g., a hydration blend). With reference to FIGS. 1-7, it is noted that an emulsion containing more than one nutrient is a particular type of nutrient blend.

FIG. 1 shows a nutrient emulsion 102 including oil 104, water 106, iron 108, vitamin C 110, lecithin 112, and fiber 114.

Oil 104 may include saturated fat, unsaturated fat, or a combination thereof. Oil 104 may include oils derived from coconut, soy, canola, peanut, sesame, palm, olive, sunflower, safflower, flaxseed, avocado, hempseed, almond, or a combination thereof. In some embodiments, the emulsion 102 includes oil 104 at a mass ratio of at least 3%, at least 10%, or at least 50%. In some embodiments, the emulsion 102 includes oil 104 at a mass ratio of at least 4%. It will be understood that the mass ratio of oil 104 affects the stability of nutrient emulsion 102 and the required mass ratios of additional elements (i.e., lecithin 112 and fiber 114) used for stabilization. In some embodiments, oil 104 is present at a mass ratio that is the balance of the other components of emulsion 102 (i.e., oil 104 is present at a mass ratio that is equal to 100% less the sum of the mass ratios of each other component of emulsion 102), before or after accounting for impurities.

Water 106 may include tap water, deionized water, reverse osmosis water, distilled water, or demineralized water. It will be understood that a type of water may affect the stability of nutrient emulsion 102 and the required quantities of stabilizing elements (i.e., lecithin 112 and fiber 114). The type of water may affect the stability of nutrient emulsion 102 due to altering liquid solution properties such as pH or hardness. In some embodiments, the emulsion 102 includes water 106 at a mass ratio of 0.5% to 5%. In some embodiments, water 106 is included at a higher ratio of 5-50%. In some embodiments, nutrient emulsion 102 is formed in a continuous media of water, after which the continuous media of water is removed (e.g., by drying) and the only remaining mass of water 106 is encapsulated within an oil 104 phase of nutrient emulsion 102.

Iron 108 includes any chelated iron, iron salt, heme, or non-heme iron. In some embodiments, iron 108 is a chelated iron such as ferrous glycinate, ferric glycinate, ferrous gluconate, ferric citrate, ferric ascorbate, carbonyl iron, heme iron (e.g., heme iron polypeptide or iron protein succinylate) (e.g., derived from animal tissue), or any combination thereof. In some embodiments, emulsion 102 includes iron 108 at a mass ratio of at least 0.025%. In some embodiments, emulsion 102 includes iron 108 at a mass ratio of at least 0.1% or at least 1.0%. The water-soluble chelated iron 108 dissolves in the water 106 phase of the nutrient emulsion 102 and is encapsulated by the oil 104 phase. The oil 104 encapsulation of the iron 108 prevents ex vivo and in vivo iron oxidation and promotes in vivo iron bioavailability due to oil 104 being broken down by enzymes, microbes, and other agents in the small intense at or near the primary site of physiological iron absorption. Therefore, the emulsion 102 provides iron 108 in a nutrient package designed to release the iron 108 at the primary site of iron absorption.

Vitamin C 110 is ascorbic acid. Vitamin C 110 further improves the bioavailability of iron 108 by forming iron chelates, mainly ferric ascorbate. In some embodiments, such chelates may form during the homogenization process and reside in the water 106 phase. In some embodiments, such chelates may form in response to the dissolution of iron 108 during ingestion and/or digestion. At gastrointestinal pH, a chelated compound of iron 108 and vitamin C 110 protects iron 108 against oxidation and thus facilitates transport to the small intestine, where the iron 108 may be absorbed for physiological use. In some embodiments, the nutrient emulsion 102 includes vitamin C 110 with at least a 25× mass or molar ratio of vitamin C 110 with respect to iron 108. In some embodiments, the mass or molar ratio of vitamin C 110 may only be 10× with respect to iron 108. With these proportional mass ratios or higher proportional mass ratios of vitamin C to iron, the chelation of iron 108 by vitamin C 110 is maximized, and the bioavailability of iron 108 is therefore maximized.

Lecithin 112 and fiber 114 stabilize oil-water interfaces within the nutrient emulsion 102 particle. In some embodiments, these nutrients may also stabilize oil-water or oil-air interfaces at the exterior of the nutrient emulsion 102 particle. In some embodiments, the nutrient emulsion 102 includes at least one of lecithin 112 or fiber 114 at a mass ratio of at least 0.5%, at least 1.0%, or at least 2.0%. In some embodiments, the emulsion may include at least one of lecithin 112 or fiber 114 at a mass ratio of at least 5% or 10%. It will be understood that the quantity of lecithin 112 or fiber 114, type of lecithin 112 (e.g., soy, sunflower, egg yolk, peanut, or wheat germ lecithin) or fiber 114 (e.g., orange peel pectin, apple peel pectin, lemon peel pectin, lime peel pectin, high-methoxyl pectin, low-methoxyl pectin, amidated pectin, sugar beet pectin, gum acacia, inulin, or any one or more oligosaccharides), ratio of lecithin 112 or fiber 114 to oil 104, ratio of lecithin 112 or fiber 114 to water 106, ratio of lecithin 112 to fiber 114, and type of lecithin 112 or fiber 114, affect the emulsion stability and the required quantities of lecithin 112 and/or fiber 114 for stabilization.

In some embodiments, including those where nutrient emulsion 102 is a powder, lecithin 112 and fiber 114 may further improve the bioavailability of iron 108 by yielding a nutrient emulsion 102 that is more soluble when mixed in a ready-to-drink liquid (e.g., water, milk, plant milk, coffee, tea, juice).

In some embodiments, nutrient emulsion 102 is dried to a powder, such as to facilitate transport and diversify its usability. As a dried powder, nutrient emulsion 102 provides bioavailable iron 108 upon being mixed in potable liquid or edible food. In some embodiments, the dry powder nutrient emulsion 102 has a density of about 0.55 g/mL and an average particle size of about 80 micrometers. In some embodiments, dried power nutrient emulsion 102 is agglomerated to increase its particle size (e.g., to over 100 micrometer) and porosity. These effects may improve its ability to dissolve when mixed in potable liquid or edible food.

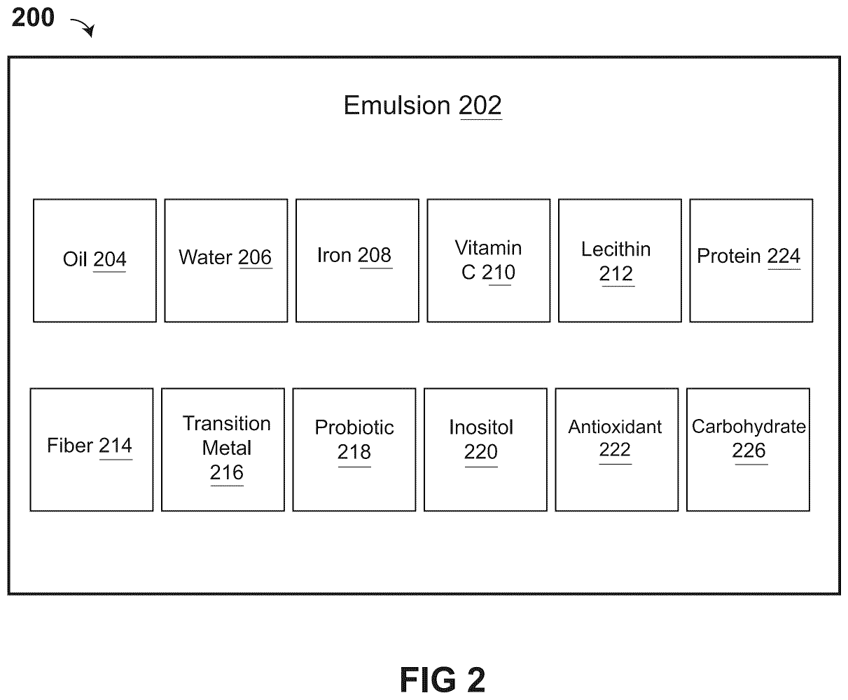

FIG. 2 shows a nutrient emulsion 202 including oil 204, water 206, iron 208, vitamin C 210, lecithin 212, fiber 214, transition metal 216, probiotic 218, inositol 220, antioxidant 222, protein 224, and carbohydrate 226, according to some embodiments of the present disclosure. Compared to the elements of nutrient emulsion 102, the added elements of nutrient emulsion 202 synergistically realize additional improvements in the bioavailability of the iron 208. In some embodiments, each of oil 204, water 206, iron 208, vitamin C 210, lecithin 212, and fiber 214, may be oil 104, water 106, iron 108, vitamin C 110, lecithin 112, fiber 114, protein 116, and carbohydrate 118, respectively.

One or more transition metal 216 (e.g., zinc, copper, cadmium, manganese, and chromium) may increase the absorption of iron 208 by activating iron transporter proteins (e.g., DMT1, FPN1) that facilitate transport of serum iron across cell membranes at iron absorption sites (e.g., the small intestine mucosal layer at the duodenum, the small intestine mucosal layer at the jejunum, the colon mucosal layer, any other iron absorption site, or any combination thereof). The one or more transition metal 216 may activate iron transporter proteins through increased expression of mRNA-encoding iron-transporter proteins (e.g., MTF-1). The one or more transition metal 216 may realize further synergistic benefits, such as supporting the immune system, suppressing infectious agents, or other benefits; these benefits may further contribute to the bioavailability of iron 208, as described in more detail below. In some embodiments, the transition metal 216 is present at a quantity of 0.05× to 0.25× the moles of the iron 208. In some embodiments, the transition metal 216 is present at 0.05× to 0.25× the mass ratio of the iron 208.

One or more probiotic 218 (e.g., B subtilis, L rhamnosus, L acidophilus, B lactis, B infantis, B breve, B longum, B bifidum, S salivarius) may increase the bioavailability of iron 208 by regulating the gut microbiome (e.g., microflora) and related processes (e.g., appetite, hormone regulation, neurotransmitter expression), supporting the immune system, suppressing infectious agents, proliferating colonic flora that digest fats at a secondary iron absorption site (e.g., the colon), or any combination thereof. The one or more probiotic 218 regulate the gut microbiome by suppressing pathogenic microbes (e.g., by outcompeting them for metabolites) and supporting beneficial microbes (e.g., by generating metabolites preferred by beneficial microbes, such as the probiotics 218 and other microbes). The one or more probiotics 218 further synergistically interact with the one or more fibers 214, wherein the latter serve as prebiotic nutrients that are digestible by probiotic microbes, including the one or more probiotics 218. The fiber helps to seed and proliferate colonies of beneficial microbes. In some embodiments, the emulsion 202 may comprise at least 1 e9 CFU of the one or more probiotic 218. Immune support due to the one or more probiotic 218 may improve the bioavailability of iron 208 by suppressing an immune response wherein pathogenic microbes (which require iron in their physiology) are starved out by way of reduced in vivo iron availability. In some embodiments, nutrient emulsion 202 is dried into a powder and probiotic 218 is dry blended into the dried powder. In some embodiments, probiotic 218 is present at a quantity of at least 1 e9 colony forming units (CFUs).

Inositol 220 increases the bioavailability of iron 208 by anti-inflammatory action (e.g., suppression of IL-6 or IL-22), by suppressing hepcidin levels (e.g., by suppressing HAMP signaling, e.g., by forming compounds with growth factors such as PI3K), or by a combination thereof. Inositol 220 may further increase the bioavailability of iron 208 by regulating hormonal cycles, including hepcidin expression, and by regulating digestive processes. Inositol may include myo-inositol, d-chiro-inositol, inositol hexaphosphate, inositol triphosphate, inositol nicotinate, or any combination thereof. In some embodiments, inositol 220 respectively includes myo-inositol and d-chiro-inositol, where the former is at a 40× mass ratio with respect to the latter. In some embodiments, inositol 220 may further mitigate symptoms of hormonal dysregulation, including PCOS. In some embodiments, inositol 220 is present at a mass ratio of at least 1.0%, at least 4.0%, or at least 10%.

One or more antioxidant 222 (e.g., glutathione, methoxatin, taurine, carnitine, lutein, glutamine, docosahexaenoic acid, lycopene, carotene, and resveratrol) increase the bioavailability of iron 208 by anti-inflammatory action (e.g., suppression of IL-1, IL-6, or IL-22), including by reducing gastrointestinal and musculoskeletal inflammation. Inflammatory cytokines such as IL-1, IL-6, and IL-22 reduce iron absorption, including by upregulating expression of hepcidin. Therefore, suppressing these cytokines may increase the bioavailability of iron 208. The one or more antioxidants 222 further serve to reduce pain as is caused by low iron and/or anemia. In some embodiments, the antioxidant 222 is present at a mass ratio of at least 0.1%, at least 1.0%, or at least 4.0%.

Protein 224 (e.g., isolates or concentrates of casein, whey, collagen, soy protein, rice protein, pea protein, bean protein, legume protein, hemp protein, egg protein, and seed protein) and carbohydrate 226 (e.g., sucrose, allulose, mogrosides, erythritol, human milk oligosaccharides, short-chain fructooligosaccharides, long-chain fructooligosaccharides, galactooligosaccharides, xylooligosaccharides, and isomaltooligosaccharides) may result in nutrient emulsion 202 having a macronutrient composition of a complete meal (i.e., all macronutrient groups are included). The complete macronutrient composition may improve the bioavailability of iron 208 by stimulating a complete set of metabolic initiation pathways, including metabolic absorption pathways of iron 208. Protein 224 may further improve the bioavailability of iron 208 by providing essential amino acids that are involved in the storage of iron 208 as ferritin. Carbohydrate 226 may further improve the bioavailability of iron 208 by stabilizing at least one of the interface (e.g., external or internal) of the emulsion through the addition of “wall” material (i.e., material that can bind to elements of an oil-water or oil-air interface). In some embodiments, protein 224 and carbohydrate 226 are each included in nutrient emulsion 202 at a mass ratio of at least 15%. In some embodiments, protein 224 is included in nutrient emulsion 202 at a mass ratio of at least 50%, and carbohydrate 226 is included in nutrient emulsion 202 at a mass ratio of at least 20%.

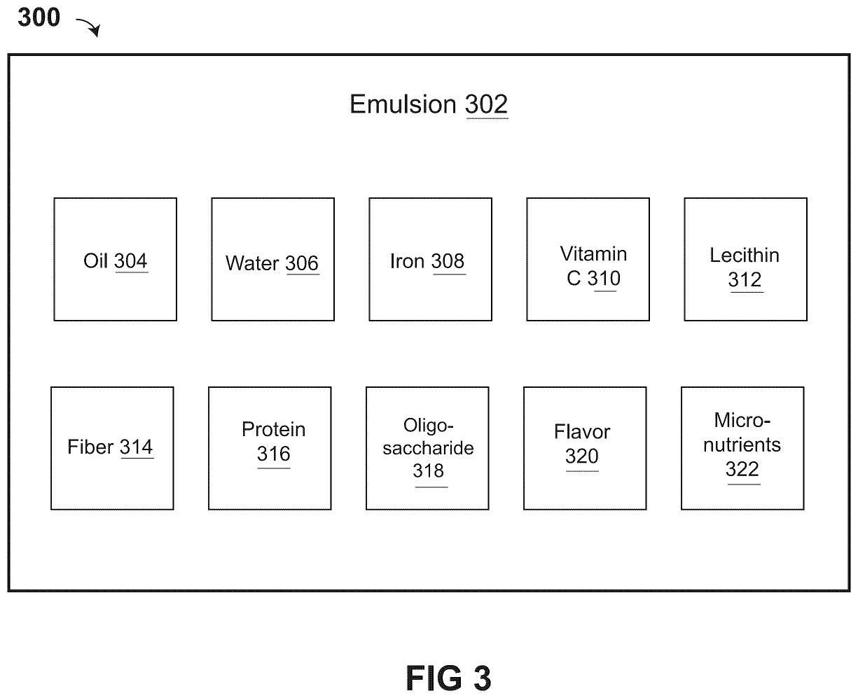

FIG. 3 shows a nutrient emulsion 302 including oil 304, water 306, iron 308, vitamin C 310, lecithin 312, fiber 314, protein 316, oligosaccharide 318, flavor 320, and micronutrients 322, according to some embodiments of the present disclosure. In some embodiments, emulsion 302 represents either of emulsion 202 or 102, plus additional elements. Compared to the elements of nutrient emulsion 102 and 202, the added elements of nutrient emulsion 302 result in a nutrient profile that is even more suitable as a meal replacement (i.e., all essential macronutrients and some or all essential micronutrients are present). In some embodiments, each of oil 304, water 306, iron 308, vitamin C 310, lecithin 312, fiber 314, protein 316, and oligo-saccharide 318 or flavor 320 may be oil 104, water 106, iron 108, vitamin C 110, lecithin 112, fiber 114, protein 226, or carbohydrate 228, respectively. In some embodiments, these quantities are adjusted in response to how the combination of all the components of emulsion 302 influences the bioavailability of iron 308 and/or the stability of emulsion 302. For example, mass ratios described in connection with nutrient emulsion 102 or nutrient emulsion 202 may be reduced based on the additional mass of the added elements of nutrient emulsion 302.

Oligosaccharide 318 (e.g., fructooligosaccharides, galactooligosaccharides, human milk oligosaccharides, xylooligosaccharides, isomaltooligosaccharides, or any combination thereof) provides prebiotic fiber that is supplemental to fiber 314 (e.g., at least one of gum or pectin). In some embodiments, the oligosaccharide 318 delivers more mass of prebiotic fiber than fiber 314. In some embodiments, oligosaccharide 318 provides soluble fiber. The fiber from oligosaccharide 318 may provide metabolites for the same probiotic microbes as fiber 314, and it may provide metabolites for other probiotic microbes. In some embodiments, oligosaccharide 318 improves the bioavailability of iron 308 due to supporting probiotic microbes, as discussed above and as further discussed below. In some embodiments, oligosaccharide 318 is present at a mass ratio of at least 5% or at least 10%.

Flavor 320 (e.g., sucrose, allulose, vanilla, cocoa, berry, orange, cinnamon, sugar, allulose, citrus, mint, monk fruit extract, erythritol, or honey) may provide a carbohydrate (e.g., one or more of carbohydrate 226) and may improve the taste of nutrient emulsion 302, which results in improved tolerance of the emulsion after ingestion. If flavor 320 includes a carbohydrate, it may provide stabilization to the nutrient emulsion. In some embodiments, flavor 320 is present at a mass ratio of less than 2% (e.g., in the absence of a carbohydrate) or at a mass ratio of at least 20% (e.g., in the presence of a carbohydrate).

Micronutrients 322 (e.g., vitamin A, vitamin D, vitamin E, vitamin K, thiamin, riboflavin, niacin, vitamin B6, vitamin B9, vitamin B12, biotin, pantothenic acid, choline, calcium, phosphorus, iodine, selenium, potassium, one or more transition metal 216, one or more probiotic 218, inositol 220, one or more antioxidant 222, or any combination thereof) promote absorption of iron 308, provides treatment of nutrition deficiencies that often occur with iron deficiency, and provide additional physiological benefits. For example, vitamins B9 and B12 are involved in erythropoiesis, such that deficiencies in those vitamins can resemble or contribute to iron deficiency. In some embodiments, micronutrients 322 improve the bioavailability of iron 308 due to stimulating its absorption (e.g., transition metals), stimulating its use (e.g., vitamins B9 and B12), or addressing other factors related to malabsorption (e.g., probiotic 218, inositol 220, antioxidant 222), as further described below. In some embodiments, folic acid, cobalamin, cholecalciferol (as a source of vitamin D), tocopheryl acetate (as a source of vitamin E), selenium, and magnesium are respectively present at quantities of 0.022×, 0.00044×, 0.0011×, 1.1×, 0.0016×, and 8.3× the mass ratio of the iron 308.

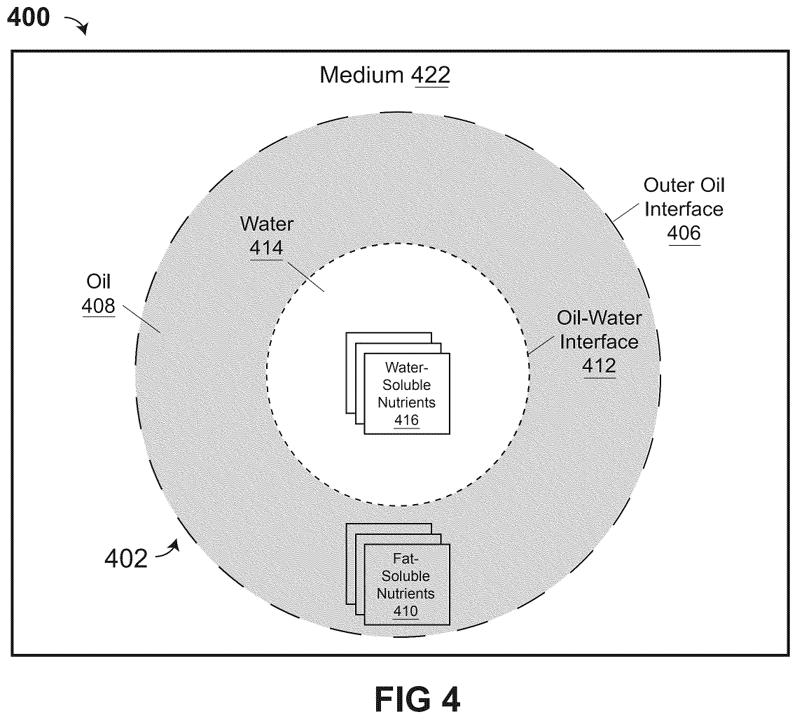

FIG. 4 shows representative elements of nutrient emulsion 402, according to some embodiments of the present disclosure. Describing FIG. 4 inward from the exterior of nutrient emulsion 402, the particle contains an outer oil interface 406, an oil phase 408 (in which fat-soluble nutrients 410 are dissolved), an oil-water interface 412, and a water phase 414 (in which water-soluble nutrients 416 are dissolved). In some embodiments, emulsion 402 may be any one of emulsion 102, 202, or 302, or related embodiments thereof. The oil-water interface 412 is stabilized by at least one of a lecithin (e.g., lecithin 112) or a fiber (e.g., fiber 114), and may be further stabilized by a carbohydrate (e.g., carbohydrate 226). Though not shown, in some embodiments, additional phases and interfaces exist (e.g., a second oil-water interface may exist within water phase 414 and across from a second oil phase, within which a third oil-water interface may exist across a second water phase, and so on).

The outer oil interface 406 is in contact with medium 422 (e.g., water, air, potable liquid, vacuum, gastrointestinal fluid, or other fluids) and includes lecithin 112, 212, or 312, fiber 114, 214, or 314, carbohydrate 226, flavor 320, or any combination thereof. In an illustrative example, when medium 422 is water, outer oil interface 406 stabilizes particles of oil 408, which are otherwise insoluble in a water medium. In another illustrative example, when medium 422 is air, outer oil interface 406 prevents oxidation of oil 408, fat-soluble nutrients 410, and water-soluble nutrients 416. Outer oil interface 406 similarly prevents all the constituent materials of emulsion 402 from releasing into medium 422.

Oil 408 releases bioavailable iron upon being metabolized by enzymes or microbes in the small intestine, which contains the primary iron absorption site. This process releases water-soluble nutrients 416 (e.g., iron, vitamin C, protein) and similarly releases fat-soluble nutrients 410 (e.g., vitamins A, D, E, and K). In some embodiments, oil 408 may be oil 104, 204, or 304.

In response to the breakdown of oil 408, water 414 releases water-soluble nutrients 416, including iron 108, 208, or 308, vitamin C 110, 210, or 310, transition metal 216, inositol 220, antioxidants 222, micronutrients 322, or any combination thereof. In some embodiments, multiple water 414 particles are present inside a single oil 408 particle. In some embodiments, additional oil particles that are similar to (but smaller than) oil 408 exist inside one or more water 414 particles. Those oil particles may encapsulate additional water particles that are similar to (but smaller than) water particle 414.

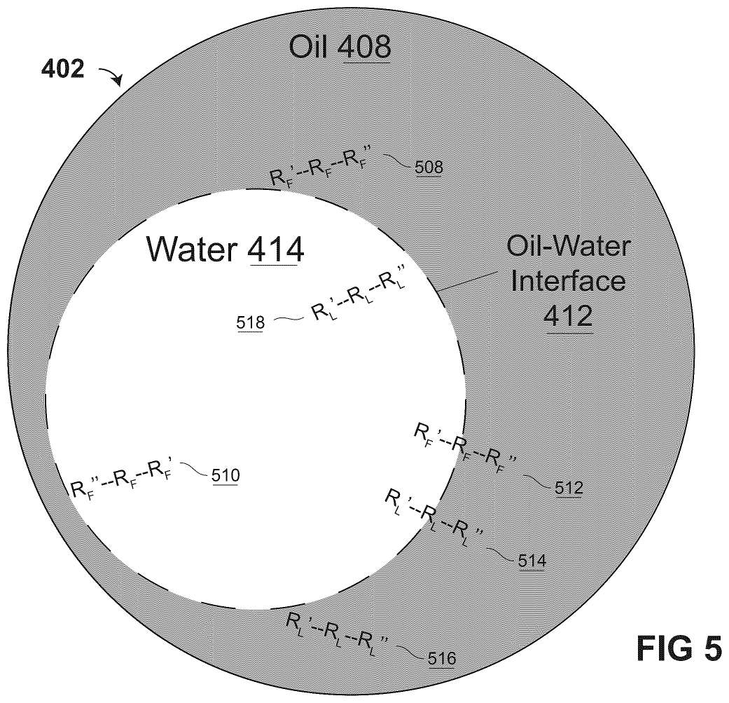

FIG. 5 shows stabilizing elements of oil-water interface 412, according to some embodiments of the present disclosure. For illustrative purposes, water particle 414 is displayed as being off-center with respect to oil particle 408. The oil-water interface 412 is either traversed or abutted by stabilizing molecules of lecithin 112, 212, or 312 (subscript ‘L’) and/or fiber 114, 214, or 314 (subscript ‘F’). Each lecithin or fiber molecule contains at least three respective functional groups (R) that are linked by chemical bonds (—). Among these functional groups is a water-soluble group (i.e., R′), an oil-soluble group (i.e., R″), and a linking group (i.e., R).

FIG. 5 shows how certain stabilizing molecules—including molecules 512 or 514—traverse the oil-water interface 412. Within these molecules, the water-soluble group may be dissolved in the water 414 phase, the oil-soluble group may be dissolved in the oil 408 phase, and the linking group may traverse the oil-water interface 412. FIG. 5 further shows how certain other stabilizing molecules—including molecules 508, 510, 516, and 518—may abut the oil-water interface. Within these molecules, the group nearest to oil-water interface 412 may be soluble in the phase across the interface and the group furthest from oil-water interface 412 may be soluble in the other phase. For example, stabilizing fiber molecule 508 abuts oil-water interface 412 and is oriented such that the water-soluble group R_{F}′ abuts the interface while the oil-soluble group R_{F}″ is dissolved in the oil 408.

In FIG. 5, though certain respective functional groups of the stabilizing molecules are shown as spanning or being directly contiguous to oil-water interface 412, it will be understood that any respective group may reside in one or both electric double layers on either side of oil-water interface 412. In some embodiments, electrostatic or electrokinetic interactions between chemical elements of a functional group and interfacial oil- or water-phase charge result in stabilization of oil-water interface 412. Additionally, though carbohydrate molecules and functional groups are not explicitly shown in FIG. 5, it is noted that certain carbohydrates (e.g., sugar, allulose, lactose, oligofructose, oligogalactose, or other carbohydrates) may also stabilize oil-water interface 412. Such carbohydrates may have a stabilizing mechanism of action that would be illustratively depicted upon swapping a subscript (i.e., ‘F’ or ‘L’) of any molecule of FIG. 5 with a subscript ‘C’.

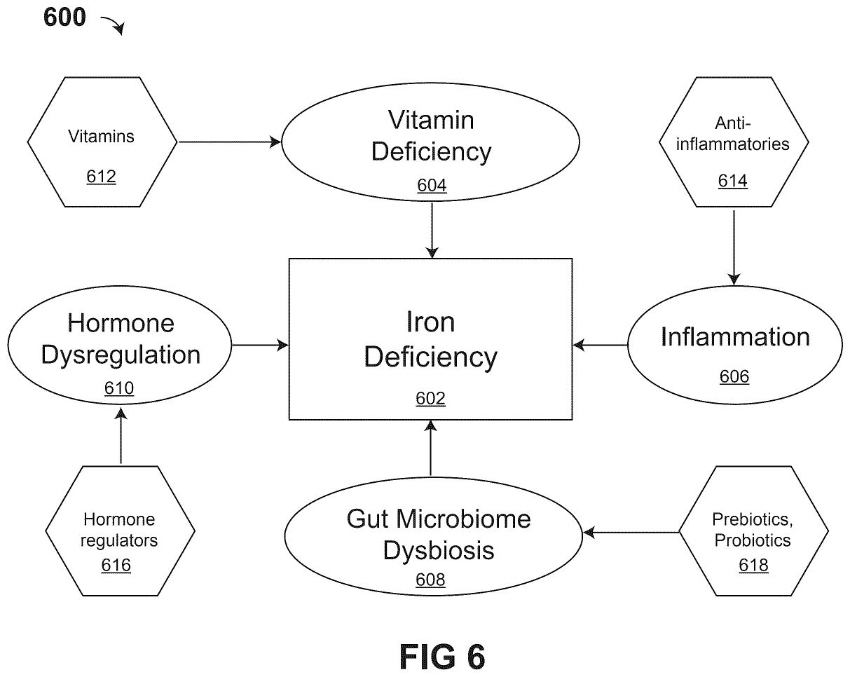

FIG. 6 shows physiological conditions contributing to iron deficiency (e.g., due to causing iron malabsorption) and corresponding nutrient-based solutions. In some embodiments, the nutrient-based solutions improve the bioavailability of iron 108, 208, or 308 and resolve iron deficiency 602 due to resolving iron malabsorption.

Vitamin deficiency 604 (e.g., deficiency of vitamins B2, B9, B12, A, C, D, E, or any combination thereof) may relate to iron deficiency 602 due to inhibiting the body from properly using the iron that is available to it. Vitamins 612 (e.g., vitamins B2, B9, B12, A, C, D, E, or any combination thereof) may be administered with iron 108, 208, or 308 to resolve vitamin deficiency 604 and thus iron deficiency 602. For example, vitamins B9 and B12 are involved in the synthesis of red blood cells, which also requires iron; vitamin B9 or B12 deficiency may thus result in red blood cell deficiency or malformation. In another example, vitamin C is involved in regulation of hepcidin, a hormone that regulates how iron in the body is absorbed and utilized, and in the synthesis of ferritin, a hormone that stores iron; vitamin C deficiency may thus result in dysregulation of iron utilization and/or storage. In another example, vitamin E is involved in protection of red blood cells through antioxidative action; vitamin E deficiency may thus result in a compromised lifetime of blood cells. In another example, vitamin B2 is involved in regulating the absorption and synthesis of iron; vitamin B2 deficiency may thus compromise iron absorption and utilization mechanisms.

Inflammation 606 may relate to iron deficiency 602 due to generating an immune response (e.g., expression of anti-inflammatory cytokines) that reduces iron availability and/or absorption. This effect may occur in populations that are prone to inflammation 606, including elders, individuals with GI diseases, individuals with other chronic diseases, and runners or high-intensity athletes. Anti-inflammatories 614 (e.g., glutathione, methoxatin, taurine, carnitine, lutein, glutamine, docosahexaenoic acid, lycopene, carotene, resveratrol, inositol, any other antioxidant, or any combination thereof) may be antioxidants administered with iron 108, 208, or 308 to resolve inflammation 606 and thus iron deficiency 602. For example, interleukins IL-1, IL-6, and IL-22 are generated in response to inflammation and promote the production of hepcidin, which inhibits iron absorption. In particular, IL-22 is related to GI inflammation. In some embodiments, anti-inflammatories 614 may therefore also include prebiotic nutrients (e.g., fiber 114, 214, or 314 or oligosaccharide 318), which may further act as an anti-inflammatory suppressing inflammatory markers generated due to gastrointestinal inflammation.

Gut microbiome dysbiosis 608 may relate to iron deficiency 602 due to generating an immune response (e.g., to suppress pathogenic microbes or other pathogens) that reduces iron availability. This immune response may be a mechanism to starve out the pathogenic microbes by slowing their natural metabolic activities that require iron. For example, sepsis conditions and viral or microbial infections induce iron deficiency 602 and may be resolved in whole or part by prebiotics and probiotics 618. Prebiotics (e.g., gum acacia, pectin, inulin, medium-chain triglycerides, oligosaccharides, or any combination thereof) and probiotics (e.g., B subtilis, L rhamnosus, L acidophilus, B lactis, B infantis, B breve, B longum, B bifidum, S salivarius) may be administered with iron 108, 208, or 308 to resolve gut microbiome dysbiosis 608 and thus iron deficiency 602. For example, prebiotics are preferentially metabolized by probiotic microbes, allowing these microbes to metabolically outcompete pathogenic microbes and repair gut microbiome dysbiosis 608. In another example, probiotics (e.g., B subtilis, L rhamnosus, L acidophilus, B lactis, B infantis, B breve, B longum, B bifidum, S salivarius) increase the gut colonization of these microbe populations and similarly allow them to metabolically outcompete pathogenic microbes and repair gut microbiome dysbiosis 608.

Hormone dysregulation 610 may relate to iron deficiency 602 due to disrupting normal levels of hepcidin (which regulates iron absorption) and/or erythropoietin (which regulates hepcidin production). Hormone regulators 616 (e.g., inositol, docosahexaenoic acid, selenium, magnesium, vitamin D) may be administered with iron 108, 208, or 308 to resolve hormone dysregulation 610 and thus iron deficiency 602. For example, inositol is a component of growth factors that signal for phosphatidylinositol-4,5-bisphosphate 3-kinase (PI3K) production and PI3K suppresses hepcidin levels via suppressing HAMP signaling.

In some embodiments of the present disclosure, nutrition compositions are provided as in Table 1.

TABLE 1
An emulsion for bioavailable iron

| Component | Mass Ratio |
|---|---|
| Coconut Oil | 40% |
| Safflower Oil | 40% |
| Soy Lecithin | 5% |
| Gum acacia | 5% |
| Ascorbic Acid | 5% |
| Water | 4% |
| Ferric Glycinate | 1% |

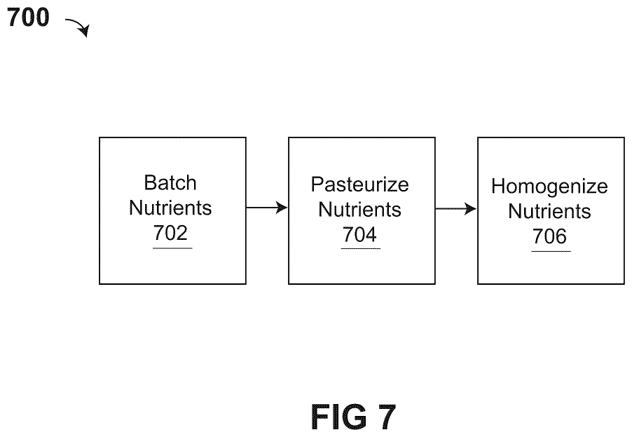

FIG. 7 shows a method for producing nutrient emulsions with improved iron absorption, in accordance with some embodiments of the present disclosure. At step 702, nutrients are batched. In some embodiments, the nutrients include those of emulsion 102, 202, or 302 or any of the nutrient blends listed below. In some embodiments, batching includes mixing nutrients in a medium such as water. At step 704, nutrients are pasteurized. In some embodiments, pasteurization is done so as to destroy microbes without damaging functional nutrients (e.g., by high-temperature short-time pasteurization, or by longer pasteurizations at lower temperatures). At step 706, nutrients are homogenized. In some embodiments, homogenization occurs upon application of high pressure and/or passage through specialized valves. For example, water 414 may become encapsulated by oil 408 during a homogenization step. In some embodiments, after step 706, the nutrients are spray dried into a powder. For example, such spray dried powder may have a bulk density of approximately 0.55 g/mL and an average particle size of 80 micrometers. In some embodiments, after spray drying, the nutrients are agglomerated into larger and more porous particles. For example, such agglomerated powder may have an average particle size of 100, 120, 150, or 200 micrometers. In some embodiments, functional ingredients including flavors 320, lecithin 112, 212, or 312, and/or fiber 114, 214, or 314, may be incorporated into a nutrient emulsion during an agglomeration step. In some embodiments, after spray drying or after spray drying and agglomerating, the nutrients may be dry blended. For example, probiotic 218 may be incorporated into a nutrient emulsion during a dry blending step.

In some embodiments, iron is provided with electrolytes (e.g., minerals), vitamins, and/or antioxidants. In this way, the iron can be administered (e.g., within an electrolyte blend) as part of a regular (e.g., used once or more each day) routine for maintaining adequate hydration (e.g., through ingestion of a hydration blend that includes the electrolyte blend). For example, such a formulation may be desirable for individuals who struggle to maintain sufficient hydration and who struggle to maintain sufficient iron levels, including but not limited to: elderly populations, people on long-term medication, people with cancer, people with GI, kidney, heart, endocrine, or liver disorders, people with nutrient malabsorption, bariatric patients, and high-performance athletes (e.g., endurance runners or bikers).

When administering iron within a group of electrolytes, it may be particularly desirable to use a ferric form of iron (e.g., ferric glycinate). The ferric form may be preferred because of not having to compete for absorption with other divalent ions (as would ferrous iron), including magnesium, calcium, copper, or zinc (which may be administered as part of the electrolyte blend). This competition is avoided because, rather than having to competitively diffuse to the apical cell membranes near the divalent metal transporter 1 (DMT1) (which is used to ingest iron and other divalent metal ions into cells), ferric iron can be reduced by duodenal cytochrome B (DCYTB), and DYCTB is typically near DMT1 on the apical membrane. In effect, a “handoff” of sorts may occur between DYCTB and DMT1, such that ferric iron is absorbed without directly competing for absorption with other divalent metal ions (e.g., as may be present in the electrolyte blend). However, in some embodiments, ferrous glycinate or other ferrous iron may be used for other reasons (e.g., cost, availability, or an absence of competitive divalent ions). In some embodiments, heme iron may be used alone or in combination with non-heme iron.

When administering iron within a group of electrolytes, it may also be beneficial to include vitamins that enhance the absorption and/or utilization (e.g., incorporation of the iron into red blood cells and/or ferritin) of the iron. In some embodiments, vitamin A, vitamin C, vitamin B12, vitamin D, vitamin E, vitamin B2 (e.g., riboflavin), and vitamin B9 (e.g., folate) (e.g., as folic acid, folinic acid, or methylfolate) may all support the absorption and/or utilization of iron. Vitamin A and vitamin C may promote iron absorption through respective mechanisms of action. Vitamin A may reduce expression of hepcidin, and hepcidin (as mentioned above) decreases iron absorption. Vitamin C may chelate ferric iron (as mentioned above) and maintain its solubility in GI fluids (e.g., as are present at the duodenum and other iron absorption sites). Vitamin B12 and folate may support iron utilization. Vitamin B12 and folate are used, with iron, in the synthesis of red blood cells. Riboflavin and vitamin D may support both iron absorption and iron utilization. Riboflavin is a cofactor in the synthesis of red blood cells, and it may contribute to regulating iron absorption, ferritin levels (which are reflective of iron storage), and iron loss. Vitamin D may reduce the expression of inflammatory cytokines (e.g., including those listed above) and hepcidin (to support iron absorption), and vitamin D may be a cofactor in the synthesis of red blood cells (to support iron utilization). Vitamin E may reduce side effects of iron ingestion (e.g., side effects due to oxidative stress) and prolong the lifespan of red blood cells due to its antioxidant functions.

In some embodiments, a first electrolyte blend includes the nutrients at the mass ratios shown in Table 2. As listed in Table 2 and other tables (e.g., Tables 3-6): the “Nutrient” column lists, in respective rows, the respective nutrients of an electrolyte blend or a nutrient blend; the “Mass” column lists, in respective rows, in milligrams (mg) or grams (g), the respective mass of each nutrient in the corresponding row; the “Compound” column lists, in respective rows, the respective one or more compound that is included in the blend to provide the nutrient in the corresponding row; the “% Nutrient” column lists, in respective rows, the typical mass percentage of each nutrient in the corresponding row when delivered as the corresponding compound (e.g., the “% Nutrient” may be the molar mass of the nutrient divided by the molar mass of the compound); the “Compound Mass” column lists, in respective rows, in milligrams (mg) or grams (g), the respective mass of each compound in the corresponding row; the “Mass Ratio (%), by Compound” column lists, in respective rows, the respective mass ratio of the corresponding compound; and the “Mass Ratio (%), by Nutrient” column lists, in respective rows, the respective mass ratio of the corresponding nutrient. For example, the “Mass Ratio (%), by Nutrient” value may be equal to the “Mass Ratio (%), by Compound” value multiplied by the “% Nutrient” value. It is noted that for some nutrients, the one or more compound (e.g., as listed in the “Compound” column) is the nutrient or a specific type of the nutrient; for other nutrients, the one or more compound is the nutrient plus a chelating agent or complementary ion; and for other nutrients, the one or more compound is a molecule that includes the nutrient. As listed in Tables 2-6, it is noted that mass values are merely illustrative of a typical serving size, and these masses may be scaled up while retaining the listed mass ratios (e.g., for delivery in packaging (e.g., individual packaging) that includes multiple serving sizes). In some embodiments, the first electrolyte blend also includes tapioca maltodextrin or any other suitable anti-caking agent (e.g., at a mass ratio of less than 2%).

TABLE 2
A first electrolyte blend

| | | | | | Mass Ratio | Mass Ratio |
|---|---|---|---|---|---|---|
| | Mass | | % | Compound | (%), by | (%), by |
| Nutrient | (mg) | Compound | Nutrient | Mass (mg) | Compound | Nutrient |
| Iron | 10 | Ferric Glycinate | 18.8 | 53 | 1.08 | 0.20 |
| Magnesium | 20 | Sodium Chloride | 14.1 | 142 | 2.06 | 0.29 |
| Sodium | 470 | Sodium Chloride | 39.4 | 1193 | 24.19 | 9.53 |
| Potassium | 470 | Potassium Citrate | 12.8 | 3686 | 74.74 | 9.53 |
| Calcium | 130 | Calcium Citrate | 8.0 | 1625 | 23.64 | 1.89 |
| Zinc | 2 | Zinc Gluconate | 14.4 | 14 | 0.20 | 0.029 |
| Vitamin B12 | 0.01 | Methylcobalamin | 100 | 0.01 | 0.00015 | 0.00015 |
| Vitamin C | 160 | Ascorbic Acid | 100 | 160 | 2.33 | 2.33 |
| Vitamin D | 0.02 | Cholecalciferol | 100 | 0.02 | 0.00029 | 0.00029 |

In some embodiments, a first instance of the first electrolyte blend may only include iron, sodium, and potassium (e.g., at the masses shown above). In some embodiments, the first electrolyte blend or its first instance may be modified to include 2 mg, 5 mg, 12 mg, 15 mg, 18 mg, 20 mg, 27 mg, or 36 mg of iron, up to 1,000 mg of sodium, up to 1,000 mg of potassium, or any combination thereof. In some embodiments, the mass ratio of the iron component in the first electrolyte blend or its first instance may be 0.1%, 0.25%, 0.5%, 1%, 1.5%, 2%, 2.5%, 3%, 3.5%, 4%, 4.5%, or 5%. In some embodiments, the iron component in the first electrolyte blend or its first instance may be iron protein succinylate, heme iron polypeptide, ferrous glycinate, carbonyl iron, any other heme iron, or any combination thereof.

In some embodiments, a second instance of the first electrolyte blend may additionally include folic acid (e.g., at a mass of at least 0.4 mg), vitamin A (e.g., at a mass of at least 0.9 mg), riboflavin (e.g., at a mass of at least 1.1 mg), or any combination thereof. The second instance of the first electrolyte blend may further be modified to increase the mass of the iron to iron mass to 18 mg, 20 mg, 27 mg, or 36 mg of iron.

In some embodiments, a third instance of the first electrolyte blend may additionally include (e.g., with respect to the first electrolyte blend or the first or second instances of the first electrolyte blend) antioxidants and/or amino acids, including methoxatin (e.g., as pyrroloquinoline quinone disodium salt at a mass of at least 5 mg), coenzyme Q10 (e.g., at a mass of at least 50 mg or at least 100 mg), beta-alanine (e.g., at a mass of at least 500 mg or at least 1,000 mg), taurine (e.g., at a mass of at least 500 mg or at least 1,000 mg), carnitine (e.g., at a mass of at least 100 mg or at least 500 mg), glutamine (e.g., at a mass of at least 500 mg or at least 1,000 mg), glutathione (e.g., at a mass of at least 500 mg or at least 1,000 mg), or any combination thereof. The third instance of the first electrolytes may further be modified to increase the iron mass to 18 mg, 20 mg, 27 mg, or 36 mg of iron.

In some embodiments, a fourth instance of the first electrolyte blend may additionally (e.g., with respect to the first electrolyte blend or the first, second, or third instances of the first electrolyte blend) include selenium (e.g., at a mass of at least 0.055 mg as selenomethionine), copper (e.g., at a mass of at least 0.9 mg as copper gluconate), manganese (e.g., at a mass of at least 2.0 mg as manganese gluconate), chromium (e.g., at a mass of at least 0.025 mg as chromium picolinate), boron (e.g., at a mass of at least 1.0 mg as boron glycinate or boron gluconate), or any combination thereof. The fourth instance of the first electrolytes may further be modified to include 18 mg, 20 mg, 27 mg, or 36 mg of ion.

It is noted that elements of the first, second, third, and/or fourth instances of the first electrolyte blend may be combined without departing from the scope and spirit of the present disclosure. In some embodiments, various electrolyte blends may incorporate some (but not all) or all elements of one or more of these instances to deliver specific health benefits (e.g., to maximize iron absorption, to pair the iron absorption with a solution to one or more vitamin/mineral deficiencies, to pair the iron with rich antioxidant and/or metabolic-boosting properties, to define a specific dosage of iron that is suited for a specific degree of iron deficiency, for any other suitable medical purpose, or any combination thereof).

As described above and as recited below in the claimed embodiments of the present disclosure, a mass or a mass ratio of a given component may be stated with reference to the nutrient or with reference to the compound. It is noted that in the construction of an electrolyte or nutrient blend comprising some, but not all, of a list of tabulated nutrients (and/or when such a blend comprises modified masses or mass ratios of the tabulated nutrients), an added nutrient (e.g., magnesium) may be dependently added to a recited blend (e.g., including iron, sodium, and potassium). In such an instance where a dependently added nutrient is added to a recited blend, it is noted that the previously-recited mass ratios of components of the recited blend are modified according to the amount of the added nutrient that is added, whether or not some or all of these modified mass ratios are explicitly stated.

TABLE 3
A second electrolyte blend

| | | | | | Mass Ratio | Mass Ratio |
|---|---|---|---|---|---|---|
| | Mass | | | Compound | (%), by | (%), by |
| Nutrient | (mg) | Compound | % Nutrient | Mass (mg) | Compound | Nutrient |
| Iron | 10 | Ferric Glycinate | 18.8 | 53 | 1.04 | 0.19 |
| Sodium | 470 | Sodium Chloride | 39.4 | 1193 | 23.21 | 9.15 |
| Potassium | 470 | Potassium Citrate | 12.8 | 3686 | 71.74 | 9.15 |
| Vitamin B12 | 0.01 | Methylcobalamin | 100 | 0.01 | 0.000195 | 0.00019 |
| Vitamin C | 160 | Ascorbic Acid | 100 | 160 | 3.13 | 3.11 |
| Vitamin D | 0.02 | Cholecalciferol | 100 | 0.02 | 0.000391 | 0.00 |
| Vitamin A | 0.9 | Beta Carotene | 53 | 2 | 0.033 | 0.02 |
| Vitamin E | 20 | Tocopheryl | 87.8 | 23 | 0.44506 | 0.39 |
| | | Acetate | | | | |
| Vitamin B2 | 1.3 | Riboflavin | 100 | 1.3 | 0.03 | 0.025 |
| Vitamin B9 | 0.4 | Folic Acid | 100 | 0.4 | 0.007815 | 0.0078 |

In some embodiments, a second electrolyte blend includes the nutrients at the mass ratios shown in Table 3. The second electrolyte blend includes sodium and potassium for hydration, iron for increasing iron levels, and vitamins A, B12, C, D, E, B2, and B9 for improving the absorption and/or utilization of the iron (e.g., through at least the respective mechanisms described above). In some embodiments, the second electrolyte blend also includes tapioca maltodextrin or any other suitable anti-caking agent (e.g., at a mass ratio of less than 2%).

In some embodiments, a first instance of the second electrolyte blend may additionally include magnesium (e.g., 20 mg magnesium as magnesium glycinate) and selenium (e.g., 30 mcg selenium as selenomethionine), both of which may help overcome anemia by acting as cofactors in biological processes requiring iron.

In some embodiments, the electrolyte blend of Table 3 (or related instances thereof) may be modified to include 2 mg, 5 mg, 12 mg, 15 mg, 18 mg, 20 mg, 27 mg, or 36 mg of iron, up to 1,000 mg of sodium, up to 1,000 mg of potassium, or any combination thereof. In some embodiments, the mass ratio of the iron component may be 0.1%, 0.25%, 0.5%, 1%, 1.5%, 2%, 2.5%, 3%, 3.5%, 4%, 4.5%, or 5%. In some embodiments, the iron component may be iron protein succinylate, heme iron polypeptide, carbonyl iron, ferrous glycinate, or any combination thereof.

In some embodiments, a second instance of the second electrolyte blend may additionally include taurine (e.g., at a mass of at least 100 mg, 250 mg, 500 mg, 1,000 or 2,000 mg), carnitine (e.g., at a mass of at least 100 mg, 250 mg, 500 mg, 1,000 or 2,000 mg), histidine (e.g., at a mass of at least 100 mg, 250 mg, 500 mg, 1,000 or 2,000 mg), lysine (e.g., at a mass of at least 100 mg, 250 mg, 500 mg, 1,000 or 2,000 mg), glutamine (e.g., at a mass of at least 100 mg, 250 mg, 500 mg, 1,000 or 2,000 mg), beta-alanine (e.g., at a mass of at least 100 mg, 250 mg, 500 mg, 1,000 or 2,000 mg), methionine (e.g., at a mass of at least 100 mg, 250 mg, 500 mg, 1,000 or 2,000 mg), threonine (e.g., at a mass of at least 100 mg, 250 mg, 500 mg, 1,000 or 2,000 mg), or any combination thereof. Each of the aforementioned amino acids may improve iron absorption, at least due to increasing the acidity of GI fluid and/or chelating circulating iron to improve iron's solubility in bodily fluids. In addition, carnitine may prolong the lifespan of healthy red blood cells, thereby reducing a strain on iron levels.

In some embodiments, the mass quantities of the aforementioned electrolyte blends represent a typical single serving that is administered to a user. For example, the typical serving may be administered as a single package (e.g., a pouch or stick pack) of powder, as a recommended serving (e.g., scoop) of a bulk container of powder, mixed into water (or any other suitable liquid) (e.g., as a ready-to-drink product), or mixed into a gel (e.g., the gel also including carbohydrates and optionally other ingredients) (e.g., as a ready-to-eat product).

In some embodiments, any of the first electrolyte blend, the second electrolyte blend, the first nutrient blend, the second nutrient blend, the third nutrition blend, or related instances or embodiments thereof may be packaged individually (e.g., without additional nutrients or additional solids or liquids, excluding the possible presence of any impurities). For example, these electrolyte blends may be packaged in bulk (e.g., a collection of many servings), such as in a plastic or fiber container. Alternatively, these electrolyte blends may be packaged as individual servings, such as in a stickpack or plastic packet. In some embodiments, these electrolyte blends may be prepared as dissolvable tablets and one or more such dissolvable tablets may be packaged together in a container (e.g., a tube or a plastic packet). In some embodiments, these electrolyte blends may be prepared within ready-to-eat gels or ready-to-drink liquids. It is noted that tabulated masses are disclosed for illustrative purposes (e.g., to contextualize a typical serving size and/or to define one or more mass ratios). In some embodiments, the tabulated nutrient or electrolyte blends are the whole (e.g., greater than 90% or greater than 95%) or at least the majority of the mass of a preparation including the nutrient or electrolyte blends. In other embodiments, the tabulated nutrient or electrolyte blends are a minority of a preparation including the nutrient or electrolyte blends (e.g., an electrolyte blend may represent less than half of the mass of a hydration powder including the electrolyte blend).

In some embodiments, any of the first electrolyte blend (or an instance thereof), the second electrolyte blend (or an instance thereof), or related embodiments may be supplemented with other ingredients to modify the taste and/or nutritional profile. In some embodiments, these supplemented blends are packaged individually (with reference to the aforementioned description of individual packaging). For example, a hydration powder may include an electrolyte blend and the hydration powder may also include an acid (e.g., at least one of citric acid, malic acid, or tartaric acid), a sweetener (e.g., at least one of sugar, allulose, or monk fruit extract), a natural flavor (e.g., a natural fruit flavor or any other suitable natural flavor), methoxatin (e.g., at a mass of at least 5 mg or at least 10 mg), coenzyme Q10 (e.g., at a mass of at least 50 mg or at least 100 mg), or any combination thereof. With respect to a hydration powder that includes the first electrolyte blend (or instances thereof) or the second electrolyte blend (or instances thereof), the acid may be present at a Mass Ratio (%), by Compound of at least 10% or at least 20%, the sweetener may be present at a Mass Ratio (%), by Compound of at least 1%, at least 3%, at least 10%, at least 20%, at least 30%, or at least 40%, and the natural flavor may be present at a Mass Ratio (%), by Compound of at least 2%, at least 5%, or at least 10%. The acid may be included to enhance the perception of the sweetener and/or the natural flavor, as well as to provide energy. The sweetener may be included to improve a taste, as well as to provide energy. The natural flavor may be included to improve a taste and give a specific flavor profile. Such hydration powders may be individually packaged (in a bulk form or as a single serving), may be tabulated for dissolution in water, may be packaged after dissolution in water as a ready-to-drink containerized liquid, or may be incorporated into a gel and packaged as a ready-to-eat containerized food.

In some embodiments, iron (with or without other electrolytes) is included in a complete nutrition blend to improve its absorption, utilization, and tolerance (e.g., compared to oral administration of only iron, e.g., as a pill). For example, iron and other nutrients may be prepared as an emulsion (e.g., a wet emulsion in which the nutrients are suspended in water, or a dry emulsion that is provided as a powder after spray drying the wet emulsion).

TABLE 4
A first nutrient blend

| | | | | | Mass Ratio | Mass Ratio |
|---|---|---|---|---|---|---|
| | | | | Compound | (%), by | (%), by |
| Nutrient | Mass (g) | Compound | % Nutrient | Mass (g) | Compound | Nutrient |
| Iron | 0.01 | Iron Glycinate | 18.8 | 0.05 | 0.12 | 0.02 |
| Protein | 20 | Pea Protein | 80.0 | 25.00 | 57.03 | 45.62 |
| Fiber | 5.75 | FOS + GA | 90.0 | 6.39 | 14.57 | 13.12 |
| Fat | 2 | HO-SO | 100.0 | 2.00 | 4.56 | 4.56 |
| Carbohydrate | 10 | Allulose | 100.0 | 10.00 | 22.81 | 22.81 |
| Phospholipid | 0.17 | Sun-Lec | 43.0 | 0.40 | 0.90 | 0.39 |

In some embodiments, a first nutrient blend includes the nutrients listed in Table 4. In some embodiments, the nutrients of Table 4 are provided as the compounds listed in Table 4, where “FOS” is short for fructooligosaccharides, “GA” is short for gum acacia, “HO-SO” is short for high-oleic sunflower oil or high-oleic safflower oil, and “Sun-Lec” is short for sunflower lecithin. The mass values, compounds, and other values shown in Table 4 are merely illustrative and non-limiting. In some embodiments, the first nutrient blend also includes tapioca maltodextrin or any other suitable anti-caking agent (e.g., at a mass ratio of less than 2%).

In some embodiments, the first nutrient blend (e.g., as listed in Table 4, or as slightly modified) comprises iron at a mass ratio of at least 0.02% (e.g., where the at least 0.02% may be 0.02%, 0.04%, 0.06%, 0.08%, 0.1%, or other suitable values), protein at a mass ratio of at least 45.0%, fiber at a mass ratio of at least 13%, fat at a mass ratio of at least 2.25%, 3.5%, or 4.5%, carbohydrate at a mass ratio of at least 22.5%, and phospholipid at a mass ratio of at least 0.35%. In some embodiments, a first instance of the first nutrient blend comprises iron as ferric glycinate at a mass ratio of at least 0.11% (e.g., where the at least 0.11% may be 0.11%, 0.22%, 0.33%, 0.44%, 0.55%, or other suitable values), protein as pea protein at a mass ratio of at least 55.0%, fiber as fructooligosaccharides at a mass ratio of at least 12.0% and gum acacia at a mass ratio of at least 1.0% or at least 2.0%, fat as high-oleic sunflower oil or high-oleic safflower oil at a mass ratio of at least 2.25%, 3.5%, or 4.5%, carbohydrate as allulose or sugar at a mass ratio of at least 22.0%, and phospholipid as sunflower lecithin at a mass ratio of at least 0.8% or soy lecithin at a mass ratio of at least 0.8%. In some embodiments, the first instance of the first nutrient blend further comprises monk fruit extract (e.g., 20%, 40%, or 50% mogrosides monk fruit extract) at a mass ratio of no more than 0.5%. In some embodiments, the first instance of the first nutrient blend further comprises water (e.g., that is residually leftover during processing from a wet emulsion into a dry powder) at a mass ratio of no more than 5% or no more than 10%. In some embodiments, the first instance of the first nutrient blend further comprises medium chain triglycerides (e.g., as fractionated coconut oil) at a mass ratio of no more than 2% or no more than 4%. In some embodiments, the first instance of the first nutrient blend further comprises a flavor (e.g., vanilla, cocoa, strawberry, fudge, cookies & cream, any other suitable flavor, or any combination thereof) at a mass ratio of less than 2%.

In some embodiments, other instances of the first nutrient blend comprise different proteins, fibers, fats (e.g., oils), carbohydrates, and/or phospholipid compounds than the first instance of the first nutrient blend (e.g., at the listed “Mass Ratio (%) by Nutrient”). For example, any of the proteins, fibers, fats (e.g., oils), carbohydrates, and/or lecithins as described in reference to FIGS. 1-7 may be used with or in place of the compounds described in connection with the first nutrient blend, the second nutrient blend, the third nutrient blend, any instance thereof, or related embodiments of the disclosed nutrient blends.

In some embodiments, a second instance of the first nutrient blend comprises iron (e.g., at the “Mass Ratio (%), by Nutrient” level listed in Table 4 or at any one of the mass ratios described in connection with the first nutrient blend) as iron protein succinylate, heme iron polypeptide, any other suitable heme iron, ferrous glycinate, carbonyl iron, or any other suitable non-heme iron, or any combination thereof. This second instance of the first nutrient blend may otherwise be equivalent to the first nutrient blend or the first instance of the first nutrient blend (e.g., accounting for the different “% Nutrient” factors and correspondingly different “Mass Ratio (%), by Compound” values when comprising iron as any one of the compounds used in the second instance versus comprising iron as ferric glycinate).

In some embodiments, a third instance of the first nutrient blend comprises only iron, protein, and fat as listed in Table 4, and the third instance of the first nutrient blend further comprises one or more emulsifier, each of the one or more emulsifier at a mass ratio of no more than 2%. For example, the one or more emulsifier can be any one or more of microcrystalline cellulose, gum acacia, or a lecithin.

TABLE 5
A second nutrient blend

| | | | | | Mass Ratio | Mass Ratio |
|---|---|---|---|---|---|---|
| | | | | Compound | (%), by | (%), by |
| Nutrient | Mass (g) | Compound | % Nutrient | Mass (g) | Compound | Nutrient |
| Iron | 0.025 | Iron Glycinate | 18.8 | 0.13 | 0.30 | 0.06 |
| Protein | 20 | Pea Protein | 80.0 | 25.00 | 55.53 | 44.42 |
| Fiber | 5.75 | FOS + GA | 90.0 | 6.39 | 14.19 | 12.77 |
| Fat | 2 | HO-SO + MCT | 100.0 | 2.00 | 4.44 | 4.44 |
| Carbohydrate | 10 | Allulose | 100.0 | 10.00 | 22.21 | 22.21 |
| Phospholipid | 0.17 | Sun-Lec | 43.0 | 0.40 | 0.88 | 0.38 |
| Vitamin C | 0.6 | Ascorbic Acid | 100 | 0.60 | 1.33 | 1.33 |
| Vitamin A | 0.0009 | Beta Carotene | 53 | 0.0017 | 0.0038 | 0.0020 |
| Vitamin B2 | 0.0013 | Riboflavin | 100 | 0.0013 | 0.0029 | 0.0029 |
| Vitamin D | 0.00003 | Cholecalciferol | 100 | 0.00003 | 0.00007 | 0.00007 |
| Taurine | 0.5 | Taurine | 100 | 0.5 | 1.1 | 1.1 |

In some embodiments, a second nutrient blend includes the nutrients listed in Table 5. In some embodiments, the nutrients of Table 5 are provided as the compounds listed in Table 5, where “MCT” is short for medium chain triglycerides, and other abbreviations (e.g., those mentioned above) are carried over from those in Table 4. The mass values and other values shown in Table 5 are merely illustrative and non-limiting. In some embodiments, the second nutrient blend also includes tapioca maltodextrin or any other suitable anti-caking agent (e.g., at a mass ratio of less than 2%).

In some embodiments, the second nutrient blend (e.g., as listed in Table 5, or as slightly modified) comprises iron at a mass ratio of at least 0.02%, 0.04%, 0.06%, 0.08%, 0.1%, or 0.12%, protein at a mass ratio of at least 44.0%, fiber at a mass ratio of at least 12.5%, fat at a mass ratio of at least 4.4%, carbohydrate at a mass ratio of at least 22.0%, and phospholipid at a mass ratio of at least 0.35%. In some embodiments, a first instance of the second nutrient blend comprises iron as ferric glycinate at a mass ratio of at least 0.1% (e.g., where the at least 0.1% may be at least 0.2%, 0.3%, 0.4%, 0.5%, or 0.6%, based on the mass ratio of the iron), protein as pea protein at a mass ratio of at least 55.0%, fiber as fructooligosaccharides at a mass ratio of at least 12.0% and as gum acacia at a mass ratio of at least 1.5% or at least 2.0%, fat as high-oleic sunflower oil or high-oleic safflower oil at a mass ratio of at least 2.2% and as MCT oil (e.g., fractionated coconut oil that is enriched in C8 and C10 saturated fats) at a mass ratio of at least 1.5% or at least 2.0%, carbohydrate as allulose, sugar, or a combination thereof at a mass ratio of at least 22.0%, and phospholipid as sunflower lecithin or soy lecithin at a mass ratio of at least 0.8%. In some embodiments, the first instance of the second nutrient blend further comprises monk fruit extract (e.g., 20%, 40%, or 50% mogrosides monk fruit extract) at a mass ratio of no more than 0.5% or no more than 1.0%. In some embodiments, the first instance of the second nutrient blend further comprises water (e.g., that is residually leftover during processing from a wet emulsion into a dry powder) at a mass ratio of no more than 5% or no more than 10%. In some embodiments, the first instance of the second nutrient blend further comprises a flavor (e.g., vanilla, cocoa, strawberry, lemon, lime, berry, orange, cherry, fudge, cookies & cream, cinnamon, any other suitable flavor, or any combination thereof) at a mass ratio of less than 2.0%.

In some embodiments, other instances of the second nutrient blend comprise different proteins, fibers, fats (e.g., oils), carbohydrates, and/or phospholipid compounds than the first instance of the second nutrient blend. For example, any of the proteins, fibers, fats (e.g., oils), carbohydrates, and/or lecithins described in connection with FIGS. 1-7 may be used with or in place of the compounds described in connection with the described instances of the second nutrient blend.

In some embodiments, a second instance of the second nutrient blend comprises iron (e.g., at the “Mass Ratio (%), by Nutrient” level listed in Table 5 or at any one of the mass ratios described in connection with the second nutrient blend) as iron protein succinylate, heme iron polypeptide, any other suitable heme iron, ferrous glycinate, carbonyl iron, any other suitable non-heme iron, or any combination thereof. This second instance of the second nutrient blend may otherwise be equivalent to the second nutrient blend or the first instance of the second nutrient blend (e.g., accounting for the different “% Nutrient” factors and correspondingly different “Mass Ratio (%), by Compound” values when comprising iron as any one of the compounds used in the second instance versus comprising iron as ferric glycinate).

In some embodiments, a third instance of the second nutrient blend comprises only iron, protein, and fat as listed in Table 5, and the third instance of the second nutrient blend further comprises one or more emulsifier, each of the one or more emulsifier at a mass ratio of no more than 2%. For example, the one or more emulsifier may be any one or more of microcrystalline cellulose, gum acacia, or a lecithin.

Comparing the first nutrient blend to the second nutrient blend (and instances thereof), the second nutrient blend provides additional vitamins and an amino acid for improving iron absorption and/or utilization (e.g., by at least the aforementioned mechanisms).

TABLE 6
A third nutrient blend.

| | | | | | Mass Ratio | Mass Ratio |
|---|---|---|---|---|---|---|
| | | | | Compound | (%), by | (%), by |
| Nutrient | Mass (g) | Compound | % Nutrient | Mass (g) | Compound | Nutrient |
| Iron | 0.025 | Iron Glycinate | 18.8 | 0.13 | 0.28 | 0.05 |
| Protein | 20 | Pea Protein | 80.0 | 25.00 | 52.91 | 42.33 |
| Fiber | 5.75 | FOS + GA | 90.0 | 6.39 | 13.52 | 12.17 |
| Fat | 2 | HO-SO + MCT | 100.0 | 2.00 | 4.23 | 4.23 |
| Carbohydrate | 10 | Allulose | 100.0 | 10.00 | 21.17 | 21.17 |
| Phospholipid | 0.17 | Sun-Lec | 43.0 | 0.40 | 0.84 | 0.36 |
| Vitamin C | 0.6 | Ascorbic Acid | 100 | 0.60 | 1.27 | 1.27 |
| Vitamin A | 0.0009 | Beta Carotene | 53 | 0.0017 | 0.0036 | 0.0019 |
| Vitamin B2 | 0.0013 | Riboflavin | 100 | 0.0013 | 0.0028 | 0.0028 |
| Vitamin D | 0.00003 | Cholecalciferol | 100 | 0.000030 | 0.000063 | 0.000063 |
| Taurine | 0.5 | Taurine | 100 | 0.500 | 1.058 | 1.058 |
| Magnesium | 0.14 | Magnesium | 14.1 | 0.993 | 2.102 | 0.296 |
| | | Glycinate | | | | |
| Selenium | 0.000028 | Selenomethionine | 40.3 | 0.000069 | 0.000147 | 0.000059 |
| Potassium | 0.36 | Potassium | 52.4 | 0.687 | 1.454 | 0.762 |
| | | Chloride | | | | |
| Vitamin B12 | 0.00001 | Methylcobalamin | 100 | 0.000010 | 0.000021 | 0.000021 |
| Vitamin B9 | 0.0004 | Folic Acid | 100 | 0.000400 | 0.000847 | 0.000847 |
| Vitamin B7 | 0.00004 | Biotin | 100 | 0.000040 | 0.000085 | 0.000085 |
| Vitamin E | 0.02 | Tocopheryl | 100 | 0.020 | 0.042 | 0.042 |
| | | Acetate | | | | |
| Methionine | 0.3 | L-Methionine | 100 | 0.300 | 0.635 | 0.635 |
| Glutathione | 0.125 | L-Glutathione | 100 | 0.125 | 0.265 | 0.265 |
| Carnitine | 0.1 | L-Carnitine | 100 | 0.100 | 0.212 | 0.212 |
| Beta Alanine | 0.25 | Beta Alanine | 100 | 0.250 | 0.529 | 0.529 |
| Glutamine | 0.25 | Glutamine | 100 | 0.250 | 0.529 | 0.529 |

In some embodiments, a third nutrient blend includes the nutrients listed in Table 6. In some embodiments, the nutrients of Table 6 are provided as the compounds listed in Table 6, where abbreviations (e.g., those mentioned above) are carried over from those in Tables 4 and 5. The mass values and other values shown in Table 6 are merely illustrative and non-limiting. In some embodiments, the third nutrient blend also includes tapioca maltodextrin or any other suitable anti-caking agent (e.g., at a mass ratio of less than 2%).

In some embodiments, the third nutrient blend (e.g., as listed in Table 6, or as slightly modified) comprises iron at a mass ratio of at least 0.02%, 0.04%, 0.05%, 0.06%, 0.08%, 0.1%, or 0.12%, protein at a mass ratio of at least 42.0%, fiber at a mass ratio of at least 12.0%, fat at a mass ratio of at least 4.0%, carbohydrate at a mass ratio of at least 21.0%, phospholipid at a mass ratio of at least 0.3%, and one or more of the micronutrients (e.g., the nutrients listed in Table 6 below “phospholipid”) of the third nutrient blend. The third nutrient blend may further comprise any one or more of the micronutrients of the third nutrient blend (e.g., at a mass ratio of at least 90% of the corresponding “Mass Ratio (%), by Nutrient” value listed in Table 6) (e.g., as the corresponding compound listed in Table 6). In some embodiments, a first instance of the third nutrient blend comprises iron as ferric glycinate at a mass ratio of at least 0.1% (e.g., where the at least 0.1% may be at least 0.2%, 0.3%, 0.4%, 0.5%, or 0.6%, based on the mass ratio of the iron), protein as pea protein at a mass ratio of at least 52.0%, fiber as fructooligosaccharides at a mass ratio of at least 10.0% and as gum acacia at a mass ratio of at least 1.0% or at least 2.0%, fat as high-oleic sunflower oil or high-oleic safflower oil at a mass ratio of at least 2.2% and as MCT oil at a mass ratio of at least 1.0% or at least 2.0%, carbohydrate as allulose, sugar, or a combination thereof at a mass ratio of at least 20%, phospholipid as sunflower lecithin or soy lecithin at a mass ratio of at least 0.8%, and one or more of the micronutrients of the third blend, where the first instance of the third nutrient blend further comprises each of the one or more micronutrients of the third nutrient blend: at a mass ratio of at least 90% of the corresponding “Mass Ratio (%), by Compound” value listed in Table 6; and as the corresponding “Compound” listed in Table 6. In some embodiments, the first instance of the third nutrient blend further comprises monk fruit extract (e.g., 20%, 40%, or 50% mogrosides monk fruit extract) at a mass ratio of no more than 0.5% or no more than 1.0%. In some embodiments, the first instance of the third nutrient blend further comprises water (e.g., that is residually leftover during processing from a wet emulsion into a dry powder) at a mass ratio of no more than 5% or no more than 10%. In some embodiments, the first instance of the third nutrient blend further comprises a flavor at a mass ratio of less than 2.0%.

In some embodiments, other instances of the third nutrient blend comprise different proteins, fibers, fats (e.g., oils), carbohydrates, and/or phospholipid compounds than the first instance of the second nutrient blend. For example, any of the proteins, fibers, fats (e.g., oils), carbohydrates, and/or lecithins described in connection with FIGS. 1-7 may be used with or in place of the compounds described in connection with the first instance of the third nutrient blend.

In some embodiments, a second instance of the third nutrient blend comprises iron (e.g., at the “Mass Ratio (%), by Nutrient” level listed in Table 6 or at any one of the mass ratios described in connection with the third nutrient blend) as iron protein succinylate, heme iron polypeptide, any other suitable heme iron, ferrous glycinate, carbonyl iron, or any other suitable non-heme iron, or any combination thereof. This second instance of the third nutrient blend may otherwise be equivalent to the second nutrient blend or the first instance of the third nutrient blend (e.g., accounting for the different “% Nutrient” factors and correspondingly different “Mass Ratio (%), by Compound” values when comprising iron as any one of the compounds used in the second instance versus comprising iron as ferric glycinate).

In some embodiments, a third instance of the third nutrient blend comprises only iron, protein, and fat as listed in Table 6, and the third instance of the third nutrient blend further comprises one or more emulsifier, each of the one or more emulsifier at a mass ratio of no more than 2%. For example, the one or more emulsifier can be any one or more of microcrystalline cellulose, gum acacia, or a lecithin. In some embodiments, the emulsifier, particularly when the emulsifier is microcrystalline cellulose, may be used as an anti-caking agent (e.g., in combination with or in place of tapioca maltodextrin). In some embodiments, the third instance of the third nutrient blend comprises the aforementioned compounds, but with a mass of iron that is doubled, tripled, or quadrupled with respect to the mass of iron as listed in Table 6.

In some embodiments of the nutrient and electrolyte blends, iron is provided as a combination of heme and non-heme iron. For example, ferric glycinate, ferrous glycinate, or carbonyl iron (e.g., non-heme irons) may be provided with iron protein succinylate, heme iron polypeptide, or other heme irons. In this way, iron absorption may be further maximized because of the discrete absorption mechanisms for heme and non-heme iron.

In some embodiments, any of the above-listed electrolyte blends or nutrient blends may substitute potassium chloride for potassium citrate.

As used herein and in the claims which follow, when a dependent claim has a preamble starting with “the X of claim N”, a transition ending with “further comprises:” or “further comprising:”, and a body including one or more components (e.g., one or more nutrient) “Y”, the dependent claim shall mean that the one or more components Y are introduced to the composite of components of the claim from which the dependent claim depends. For example, the one or more components Y are added to the composition X, or alternatively are added to a composition W (e.g., when the composition X comprises the composition W). It is noted that in such a construction, the mass ratio(s) of the one or more components Y are recited based on the presence of this component(s) in the composite mixture X+Y (or W+Y). Unless explicitly stated otherwise, the mass ratios of the components of X (or W), e.g., as recited in claim N, are reduced in the composite mixture X+Y (or W+Y). The magnitudes of these reductions are based on the cumulative amount of the one or more components Y that are added. This trend is extended in the case of a composite mixture X+Y+Z (or W+Y+Z), and so on (e.g., W+Y+Z+ . . . ), where “Z” represents one or more components that are introduced, after “Y”, in the same chain of dependency and “ . . . ” represents additional components that are similarly introduced after “Z”. In other words, if a dependent claim recites that the claim from which the dependent claim depends “further comprises:” (or is “further comprising:”) an added component, then the total composite mass (inclusive of the mass recited in the chain of dependency) of the recited dependent claim is necessarily greater than the total composite mass of the recited matter of the claim from which the respective claim depends.

It is noted that the foregoing is only illustrative of the principles of the invention, and that the invention can be practiced by other than the described embodiments, which are presented for purposes of illustration and not limitation, and the present invention is limited only by the claims which follow.

What is claimed is:

1. A hydration powder comprising an electrolyte blend, the electrolyte blend comprising:
an iron component present at a mass ratio of at least 0.15%;
a sodium component present at a mass ratio of at least 9.0%; and
a potassium component present at a mass ratio of at least 9.0%.

2. The hydration powder of claim 1, wherein:
the iron component comprises ferric glycinate, the ferric glycinate at a mass ratio of at least 1.05%;
the sodium component comprises sodium chloride, the sodium chloride at a mass ratio of at least 24.1%; and
the potassium component comprises potassium citrate, the potassium citrate at a mass ratio of at least 74.7%.

3. The hydration powder of claim 2, wherein the electrolyte blend further comprises:
a magnesium component present at a mass ratio of at least 0.29%;
a calcium component present at a mass ratio of at least 1.8%;
a zinc component present at a mass ratio of at least 0.029%;
a methylcobalamin component present at a mass ratio of at least 0.00014%;
a cholecalciferol component present at a mass ratio of at least 0.00028%; and
an ascorbic acid component present at a mass ratio of at least 2.3%.

4. The hydration powder blend of claim 3, wherein:
the magnesium component comprises magnesium glycinate, the magnesium glycinate at a mass ratio of at least 2.0%;
the calcium component comprises calcium citrate, the calcium citrate at a mass ratio of at least 23.5%; and
the zinc component comprises zinc gluconate, the zinc gluconate at a mass ratio of at least 0.15%, such that:
the mass ratio of the ferric glycinate is reduced to at least 0.7%,
the mass ratio of the sodium chloride is reduced to at least 17.0%, and
the mass ratio of the potassium citrate is reduced to at least 53.0%.

5. The hydration powder of claim 1, wherein the electrolyte blend further comprises:
a methylcobalamin component present at a mass ratio of at least 0.00019%;
a cholecalciferol component present at a mass ratio of at least 0.00039%;
an ascorbic acid component present at a mass ratio of at least 3%;
a beta carotene component present at a mass ratio of 0.3%;
a tocopheryl acetate component present at a mass ratio of at least 0.4%;
a riboflavin component present at a mass ratio of at least 0.02%; and
a folate component present at a mass ratio of at least 0.007%.

6. The hydration powder of claim 5, wherein the electrolyte blend further comprises:
a taurine component present at a mass ratio of at least 0.18%; and
a carnitine component present at a mass ratio of at least 0.18%.

7. The hydration powder of claim 1, further comprising:
at least one acid, the at least one acid comprising at least one of citric acid, malic acid, or tartaric acid;
at least one sweetener, the at least one sweetener comprising at least one of sugar, allulose, or monk fruit extract;
at least one natural flavor; and
methoxatin.

8. A packaged electrolyte blend comprising:
an iron component present at a mass of at least 2 mg;
a sodium component present at a mass of at least 400 mg; and
a potassium component present at a mass of at least 400 mg.

9. The packaged electrolyte blend of claim 8, wherein the packaged electrolyte blend is individually arranged within a sealed container.

10. The packaged electrolyte blend of claim 9, wherein the sealed container further comprises: at least 6 ounces of water.

11. The packaged electrolyte blend of claim 9, wherein the sealed container further comprises: at least 1 ounce of gel.

12. The packaged electrolyte blend of claim 11, wherein the at least 1 ounce of gel comprises the iron component, the sodium component, the potassium component, and a carbohydrate component.

13. The packaged electrolyte blend of claim 8, wherein the packaged nutrient blend is individually arranged as a dissolvable tablet.

14. The packaged electrolyte blend of claim 8, wherein the iron component is present at a mass of at least 10 mg, wherein the iron component comprises at least one of ferric glycinate or ferrous glycinate.

15. The packaged electrolyte blend of claim 8, wherein the iron component is present at a mass of at least 10 mg, wherein the iron component comprises at least one of iron protein succinylate or heme iron polypeptide.

16. The packaged electrolyte blend of claim 8, further comprising:
a methylcobalamin component present at a mass ratio of at least 0.01 mg;
a cholecalciferol component present at a mass ratio of at least 0.02 mg; and
an ascorbic acid component present at a mass ratio of at least 160 mg.

17. The packaged electrolyte blend of claim 16, further comprising:
a beta carotene component present at a mass of at least 0.9 mg;
a tocopheryl acetate component present at a mass of at least 20 mg;
a riboflavin component present at a mass of at least 1.3 mg; and
a folate component present at a mass of at least 0.4 mg.

18. The packaged electrolyte blend of claim 8, further comprising:
a taurine component present at a mass of at least 100 mg;
a carnitine component present at a mass of at least 100 mg; and
a methoxatin component present at a mass of at least 5 mg.

19. The packaged electrolyte blend of claim 8, further comprising: at least one amino acid present at a mass of at least 100 mg, the at least one amino acid comprising at least one of glutamine, beta-alanine, methionine, histidine, leucine, or threonine.

20. The packaged electrolyte blend of claim 8, further comprising: at least one antioxidant present at a mass of at least 100 mg, the at least one antioxidant comprising at least one of glutathione, methoxatin, docosahexaenoic acid, lycopene, carotene, or resveratrol.
